(19) United States
(12) Patent Application Publication
(10) Pub. No.: US 2013/0150958 A1
(43) Pub. Date: Jun. 13, 2013

(54) ANNULOPLASTY BAND FOR A SIMPLIFIED APPROACH TO MITRAL VALVULOPLASTY FOR DEGENERATIVE DISEASES

(71) Applicant: Ruggero De Paulis, Rome (IT)

(72) Inventor: Ruggero De Paulis, Rome (IT)

(21) Appl. No.: 13/814,030

(22) PCT Filed: Aug. 2, 2010
(86) PCT No.: PCT/IT2010/000347

§ 371 Date: Feb. 13, 2013

(51) Int. Cl. A61F 2/24

(52) U.S. Cl. CPC A61F 2/2445
USPC 623/2.36

(57) ABSTRACT

A device for repair of heart valves, and more particularly an improved annuloplasty band which is constituted by a semirigid open band includes a sheath and a generally curved stiffening semirigid element disposed within the sheath which extends from a first end adjacent the antero-lateral trigone to a second end adjacent the postero-medial trigone defining a non flat (not coplanar) compound C-curve shape so as to mime both in anterior and posterior side a saddle shape.

The present invention relates substantially to a device for repair of heart valves, and more particularly to an improved annuloplasty band.

Annuloplasty prostheses, generally categorized as either annuloplasty rings or annuloplasty bands, are employed in conjunction with valvular reconstructive surgery to assist in the correction of heart valve defects such as stenosis and valvular insufficiency. There are two atrio-ventricular valves in the heart. That on the left side of the heart known as the mitral valve, and that on the right side known as the tricuspid valve. Anatomically speaking, each valve type forms or defines a valve annulus and valve leaflets. To this end, the mitral and tricuspid valves differ significantly in anatomy. Whereas the annulus of mitral valve is somewhat “D” shaped, the annulus of the tricuspid valve is more nearly circular.

Both valves can be subjected to or incur in damage that requires that they must be repaired or replaced. The effects of valvular dysfunction vary. Mitral regurgitation has more severe physiological consequences to the patient than tricuspid valve regurgitation, a small amount of which is tolerated quite well. Many of the defects are associated with dilation of the valve annulus. This dilation not only prevents competence of the valve but also results in distortion of the normal shape of the valve orifice. Remodeling of the annulus is therefore central to most reconstructive procedures on the Mitral valve. In this regard, clinical experience has shown that repair of the valve, when technically possible, produces better long-term results as compared to valve replacement.

Many procedures have been described to correct pathology of the valve leaflets and their associated chordae tendinae and papillary muscles. In mitral repairs, it is considered important to preserve the normal distance between the two fibrous trigones. The trigones almost straddle the anterior leaflet portion of the annulus.

A significant surgical diminution of the inter-trigonal distance could cause left ventricular outflow obstruction. Thus, it is highly desirable to maintain the natural inter-trigonal distance during and following mitral valve repair surgery.

Consequently, when a mitral valve is repaired (be it the posterior or anterior leaflet) the result is generally a reduction in the size of the posterior segment of the mitral valve annulus. As a part of the mitral valve repair, the involved segment of the annulus is diminished (i.e. constricted) so that the leaflets may coapt correctly on closing, and the annulus is stabilized to prevent post-operative dilatation from occurring. This is frequently achieved by the implantation of a prosthetic ring or band in the supra annular position. The purpose of the ring or band is to restrict and/or support the annulus to correct and/or prevent valvular insufficiency. However, it is important not to over restrict the annulus or an unacceptable valvular stenosis could result.

As described above, both annuloplasty rings and annuloplasty bands are available for repair of an atrio-ventricular valve.

A flexible annuloplasty ring has been available, among others, under the trade designation “DURAN(™)” by Medtronic, Inc., USA. In general terms, annuloplasty rings completely encompass both the anterior and posterior portions of the valve annulus. The posterior portion is often diseased or dilated and not well supported by heart tissue. The anterior portion, in contrast, is well supported by surrounding heart tissue. Thus, it is possible that the annuloplasty ring may overtly support an otherwise healthy anterior portion, potentially leading to tissue failure.

Annuloplasty bands, on the other hand, are specifically designed to primarily encompass only a portion of the valve annulus. For example, a mitral valve annuloplasty band is typically configured to encompass only the posterior portion of the mitral valve annulus, thus promoting natural movement of the anterior portion. In addition to facilitating natural movement of the healthy portion of the valve annulus, annuloplasty bands can be implanted more quickly than annuloplasty rings, as fewer sutures are required. Examples of annuloplasty bands are shown in U.S. Pat. No. 5,824,066 and WO2000/74603. While viable, annuloplasty bands present other concerns. For example, if the band is only anchored into friable valve annulus tissue, there is some concern that the band may possibly pivot excessively relative to the valve annulus. Furthermore, the profile (e.g., thickness) of prior annuloplasty bands may theoretically be sufficiently large so as to restrict or disturb blood flow.

Furthermore, from U.S. Pat. No. 7,371,259 an annuloplasty band adapted to provide reinforced attachment to the valve annulus, and a holder and a sizer that facilitate implantation thereof are known.

In the field of annuloplasty prostheses it is well known that rings or bands are needed for durable results, that any type of ring abolishes annular movements, and that intertrigonal distance does not change.

### The Saddle: Aorto-Mitral Junction

During systole annular contraction occurs, narrowing the valve orifice, the commissural areas are pulled down accentuating the anterior mitral saddle and accommodating the bulging root (Adams and coll. Curr. Cardiology Reports 2008; 10:226-32).

Aortic and mitral annular area have coupled reciprocal behavior: AoArea expansion may facilitate Mitral Area expansion and viceversa (Veronesi and coll. Circ Cardiovasc Imaging 2009; 2:24-31).

The angle between the two valves changes during the cardiac cycle supporting the role of the fibrous continuity as an anchor when the two annuli move towards each others (Veronesi and coll. Circ Cardiovasc Imaging 2009; 2:24-31)

Annulus sistolic dynamics are substantially:
Septo-lateral distance=−23%
Comm-Comm distance=−12%
Inter-trigonal distance=+11%

Importance of the saddle shape is due to a plurality of advantages: Maintains leaflet curvature decreasing leaflet and chordal stress (Ryan et al. e Ann Thorac Surg 2008; 86:749-760); Reduces stress on anterior leaflet (Jimenez JTCVS 2007; 134:1562-8; Jensen Circulation 2008; 118:S250-55) Reduces stress on both leaflets (Salgo et al. Circulation 2002; 106:711-7).

For all these reason it is important to maintain or re-established a normal saddle shape after surgery because a more normal physiology will probably make the operation of valve plasty more durable and efficient.

The present invention substantially relates to an improved annuloplasty band for mitral valve plasty having a saddle shape, thereby obtaining both the advantages of an open band and those of the saddle shape.

A better understanding of the invention will be obtained from the following detailed description with reference to the attached drawings, which illustrate, purely by way of non-limiting example, a preferred embodiment thereof.

In the drawings:

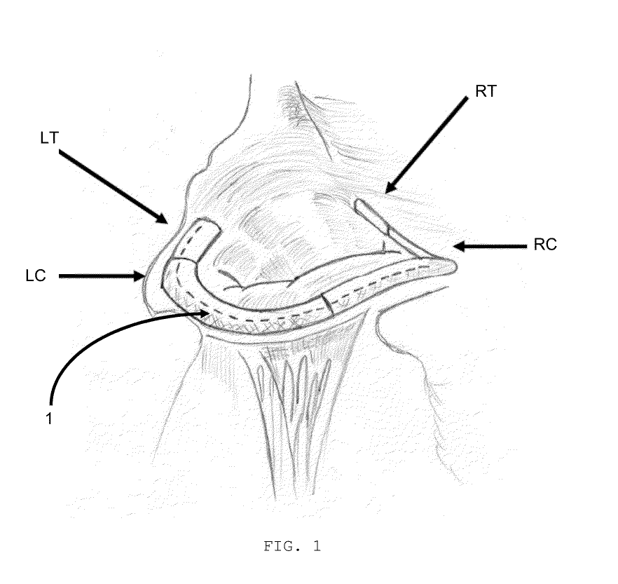

FIG. 1 is a 3D view of the open band with saddle shape according to the invention.

### ADVANTAGES OF AN OPEN BAND

The use of an Open band is important in order to: Avoid anterior leaflet folding or distortion, Avoid unnecessary suture placement, Leave enough space to the surgeon for chordal placement.

More particularly, the avoiding of unnecessary suture placement is very advantageous in the case of MICS (minimally invasive approach) and because it reduces the risk of leaflet tearing, and of interfering with the aortic root (c.f.r. reimplantation below).

Furthermore, it has to be noted that the Open ring is fast, effective, more forgiving (no folds, no tearing, no distortion), and has a larger orifice area.

On the other hand it is well established how the open band is able to achieve all the objective of closed ring and in particular:

it is known that Semirigid Band (or open ring) restores annular geometry, i.e. D-shape is restored (Redmond et al. J Heart valve Dis 2008; 17:115-8);

respects anterior motion of the saddle (Redmond J Heart valve Dis 2008; 17:115-8.Timek et al. Ann Thorac Surg 2001; 72:966-74).

the implantation of an open band gives durable results (Salvador et al. J Thorac Cardiovsc Surg 2008; 135: 1280-1287.)

### The Role of Full Ring with a Saddle Shape

Full rings with a saddle shape have been designed with the aim of re-establishes the saddle shape. However the drawbacks of such rings are that the saddle shape is fixed both in systole and in diastole and its shape can not change during the cardiac cycle. Furthermore the use of a full ring makes the anterior portion of the mitral valve rigid with time, as the rest of the annulus. In particular this portion of the ring might also interfere with the base of the aorta. In fact it has been reported that during a reimplantation procedure (a procedure used to replace the aortic root) it was necessary to remove the mitral ring because it distorted the right and noncoronary aortic cusps (Mohr in discussion of Redmond and coll.)

### Saddle Shape and Open Band

For what saddle shape and open band are concerned, it should be noted that after implant of Cosgrove band the saddle shape is restored (Timek et al. Ann Thorac Surg 2001; 72:966-74); Saddle shape is in fact the ratio between annular height to C-C (commissuro-commissural) width. When C-C is restored this ratio will obviously increase. Furthermore, it is proved that only a partial ring preserves anterior annular flexion during cardiac cycle (Dagum et al. JTCVS 2001; 122:665-73). In fact it keeps anterior MA (mitral angle) elevation and anterior posterior MA flexion.

At present the known devices for valve repair either are full rings (with or without a saddle shape) or are flat open band. There are two types of open band: one that takes their shape only after implantation (being completely souple not having any rigidity)and another one (Future-Band) that is semirigid and flat.

The known devices for heart valve repair have a plurality of drawbacks those of a closed ring (with or without a saddle shape) have been outlined but are basically related to their interference with the anterior portion of the mitral valve that is unable to maintain its movement during the cardiac cycle as well as negatively influencing the physiologic movements of the aortic root. Regarding the existing open band they:
i. Do not have a saddle shape (ant. or post.)
ii. There is not a marked portion corresponding to the commissures and another corresponding to the trigones
iii. Curvature at their ends are non-existing or are too sharp.

According to the present invention, it is provided a device constituted by an open saddle 1 (herein after will be indicated also as “Physio-C” for being physiologic with a “C” shape) as schematically shown in FIG. 1.

Advantageously, the Physio-C or open saddle 1 has an optimized shape for increasing size (from D-shape to a more circular similar to the device known as “Physio II”) and an optimized curvature at the commissures.

Furthermore, the device according to the present invention is semirigid and has an initial saddle shape at the trigones, thereby obtaining a clear location for trigones (LT and RT) and for commissures (LC and RC).

A peculiar feature of the invention, consists in that said saddle-shape is provided both to the anterior side and to the posterior side.

The two ends of the “C” shape of the open saddle 1 are rounded and with the increase of the size of the device, its general shape changes from a more oval shape to a more circular shape: in other words, when the size increases, then the antero-posterior diameter increases progressively more that the latero-lateral diameter.

Differently from known Physio II,
It does not fix the aorto-mitral junction
It allows cyclic movement of the saddle
It allows for possible posterior displacement of the saddle.

Accordingly, the open saddle 1 of the present invention preserves the saddle but does not fix it in systole: therefore, it combines the advantages of the open band and the saddle ring.

While the known Cosgrove-Edwards Annuloplasty System is completely soft, the open band with saddle shape according to the present invention comprises an inner stiffening element which maintains said curved (saddle shape) shape.

1. A device to repair heart valves, characterized in that it is constituted by a semirigid open band (1) comprising a sheath and a generally curved stiffening semirigid element disposed within the sheath which extends from a first end adjacent the antero-lateral trigone to a second end adjacent the postero-medial trigone defining a non flat (not coplanar) compound C-curve shape so as to mime both in anterior and posterior side a saddle shape.

2. A device according to claim 1 wherein the two ends of the C-shape of the open band are rounded; thereby obtaining a clear location for trigones (left trigone, LT; right trigone, RT) and for the commissures (left commissure, LC; right commissure, RC).

3. A device according to claim 1 characterized by the fact that with the increase of the size of the device, its general shape changes from a more oval shape to a more circular shape.

4. A device according to claim 1 characterized in that it has an initial saddle shape at the trigones.

5. A device according to claim 1 characterized in that markers are provided in the sheath that identify both the commissures and the trigones.

6. A device according to claim 2 characterized by the fact that with the increase of the size of the device, its general shape changes from a more oval shape to a more circular shape.

7. A device according to claim 2 characterized in that it has an initial saddle shape at the trigones.

8. A device according to claim 3 characterized in that it has an initial saddle shape at the trigones.

9. A device according to claim 2 characterized in that markers are provided in the sheath that identify both the commissures and the trigones.

10. A device according to claim 3 characterized in that markers are provided in the sheath that identify both the commissures and the trigones.

11. A device according to claim 4 characterized in that markers are provided in the sheath that identify both the commissures and the trigones.
